(19) United States
(12) Patent Application Publication
(10) Pub. No.: US 2005/0053999 A1
(43) Pub. Date: Mar. 10, 2005

(54) Method for predicting G-protein coupled receptor-ligand interactions

(76) Inventors: David A. Gough, Cardiff, CA (US); Joel R. Bock, El Cajon, CA (US)

Correspondence Address: GORDON & REES LLP, 101 WEST BROADWAY, SUITE 1600, SAN DIEGO, CA, 92101, US

(21) Appl. No.: 10/973,576

(22) Filed: Oct. 26, 2004

Related U.S. Application Data
(63) Continuation-in-part of application No. 09/993,272, filed on Nov. 14, 2001.
(60) Provisional application No. 60/248,258, filed on Nov. 14, 2000.

(51) Int. Cl. C12Q 1/68; G06F 19/00; G01N 33/48; G01N 33/50

(52) U.S. Cl. 435/6; 702/20

(57) ABSTRACT

The invention is a teachable system and method for predicting the interactions of proteins with other proteins, nucleic acids and small molecules. A database containing protein sequences and information regarding protein interactions is used to “teach” the machine. Proteins with unknown interactions are compared by the machine to proteins in the database. Homologs of proteins known to interact in the database are predicted to interact.

### CROSS-REFERENCES TO RELATED APPLICATIONS

This application claims the benefit of priority of U.S. non-provisional application Ser. No. 09/993,272272 filed Nov. 14 2001, which claims the benefit of provisional application 60/248,258 filed Nov. 14, 2000.

### COMPUTER APPENDIX

A computer program listing appendix submitted in duplicate on compact disc under §1.52 ((e) 5) with the application Ser. No. 09/993,272 s hereby incorporated by reference.

### FIELD OF THE INVENTION

The invention is a trainable system and computational method for predicting the interaction of biopolymers with other biopolymers, nucleic acids, and with a variety of ligands based on the sequence or primary structure of the biomolecule.

### BACKGROUND OF THE INVENTION

Determination of protein-protein interaction is a slow and cumbersome process. Methods such as the yeast two-hybrid system can reveal unexpected, transient protein-protein interactions in cells. Alternatively, more stable protein-protein interactions may be determined by immunoprecipitations and other in vitro binding assays. However, it is generally not possible to determine the specific sites of interaction between the proteins by these methods. High-resolution structural analysis can reveal protein-protein interactions at a molecular level. Structures can be obtained for protein complexes, but only proteins already known to interact would be studied in this manner. Pairs of proteins may be studied individually to predict protein-protein interactions, but there is no high-throughput method to search for proteins that will likely interact with a protein of interest. Even if such a method did exist, it would be limited by the number of protein structures that are available in databases.

Similarly, methods to determine protein-nucleic acid interactions and protein-ligand binding interactions are also cumbersome. A number of binding assays, both in vitro and in vivo have been developed depending on the interaction to be analyzed. Although some of these methods may be relatively high throughput, based on 96-well plates with automated read out, the process of analyzing 10,000 compounds produced by combinatorial chemistry can be daunting.

Computational prediction of interactions has involved estimation of the site of interaction, utilization of features and properties related to interface topology, solvent accessible surface area, and hydrophobicity, or the recognition of specific residue or geometric motifs. These computational methods are highly specialized, require specific physiochemical information that is generally not available for all proteins, and are not broadly applicable.

Genome projects in a variety of organisms have provided researchers with a large amount of DNA sequence information. Gene chip technology has provided a means to analyze gene expression under a variety of conditions, including development and disease. However, although genes can frequently be assigned into groups based on DNA sequence (e.g. kinases, transcription factors, structural proteins, etc), the way that the proteins interact is not revealed by DNA sequence.

Protein function is exceedingly diverse. Within the cell, proteins assemble into complex and dynamic macromolecular structures, recognize and degrade foreign molecules, regulate metabolic pathways, control DNA replication and progression through the cell cycle, synthesize other chemical species, facilitate molecular recognition, localize and “scaffold” other proteins within signal transduction cascades and participate in other important functions.

To appreciate the breadth of protein function, a description of protein-protein interactions is a necessary first step. Beginning with the proteomic constituents, a rational research strategy should then proceed in the direction of abstract information flow represented by interaction □ network □ function rather than the more typical function □ interaction □ network.

Given the volume of proteomic data generated by high-throughput technologies, prediction of protein function requires integration of empirical data with bioinformatic comparative prediction analyses. For example, a complete pairwise protein interaction in the relatively tiny proteome of the bacterium Mycoplasma genitalium, with N=486 proteins, requires screening of N(N−1) or 235,710 separate interactions (EBI Proteome Analysis database; http://www.ebi.ac.uk/proteome). The task would be overwhelming if approached by experiment alone.

The workhorse of experimental proteomics has been the two hybrid screen (Fields and Song, 1989), which has been criticized based on the accuracy of the results and its labor intensive nature (Enright et al., 1999). Protein chips may eventually provide large scale simultaneous protein-protein interaction data (MacBeath and Schreiber, 2000), but technical problems (denaturing, substrate biocompatablity) must be overcome to scale-up for high-throughput analysis. Moreover, the preparation of chips is non-trivial. As application of proteins from cell or tissue homogenates directly to the chip would not be possible as the resulting chip would be coated with predominantly structural proteins which tend to represent the plurality of cell proteins. Unlike nucleic acids that may be amplified from a chip, the small amounts of protein on a chip would be insufficient for sequencing. Therefore, proteins would need to be expressed and applied to a chip at distinct locations to allow for identification of the protein bound by the probe. An individual chip would need to be prepared for the analysis of every few protein probes depending on multiplex capacity of the system. Improved technologies are required before protein chip technology is practical and affordable.

Other approaches may become prominent as proteomics technology continues to evolve: for example, denaturing may be avoided by combining high performance liquid chromatography (HPLC) co-elution with MALDI-TOF (Matrix Assisted Laser Desorption Ionization) mass spectrometry (Champion et al, 2001). Thus, one may isolate complexes by chromatography, separate the components of the complex and identify them by sequencing then individually. Such systems do not allow for the definition of individual protein-protein interactions, but instead provide information on complexes which then must be analyzed by further experimentation to determine the individual interactions.

### SUMMARY OF THE INVENTION

The invention is a trainable system and method for the prediction of the interactions, mutual bindings or associations between specific homogenous pairings of biomolecules such as, but not limited to, protein-protein, DNA-DNA, and heterogenous pairings such as protein-DNA, protein-RNA, DNA-RNA, etc. The predictions are based on primary protein sequence available in electronic format and associated physiochemical information also available in electronic format such as hydrophobicity, charge and chemical composition.

For example, primary structure of a vast number of proteins is now available in electronic format, with associated physiochemical properties of each amino acid. These data can be digitally encoded as a sequence of numbers, this new sequence representing the properties of each protein in potential binding interaction. The trainable system is trained to recognize patterns in these sequences, specifically patterns that characterize positive interaction with between proteins as observed experimentally. This system makes a statistical decision as to whether or not a new pair of proteins will interact, based on its “training” from previous data. The system achieves a high degree of precision relative to previous methods in making these decisions, enabling higher throughput screening of potential candidate proteins for different applications.

The invention can be applied to larger scale studies of protein-protein interactions in a proteome wide scale. Application of a “phylogenetic bootstrap” method for protein-protein interaction mining, which comprises traversal of a phenogram, interleaving rounds of computation and experiment, to develop a knowledge base of protein interactions in genetically similar organisms. The steps comprising phylogenetic bootstrap are distilled into an algorithm, described herein in detail. Similar methods can be applied to predict interactions of other types of biomolecules.

### BRIEF DESCRIPTION OF THE DRAWINGS

The present invention will be better understood from the following detailed description of an exemplary embodiment of the invention, taken in conjunction with the accompanying drawings in which like reference numerals refer to like parts and in which:

FIG. 1. Scatterplot showing detail view of sample datapoints \tilde{x}ᵢ□ⁿ representing H. pylori protien-protein interactions, visualized by two dimensional Sammon mapping. Circled points indicate incorrect decisions made during leave-one-out prediction error estimation. 90% of all data points (1,873/2,077) appearl in this map. Coordinate axes contain arbitrary units. Estimated system generalization error rate is 12.04%.

FIG. 2 shows two-dimensional structures corresponding to the top nine compounds listed in Table 5.

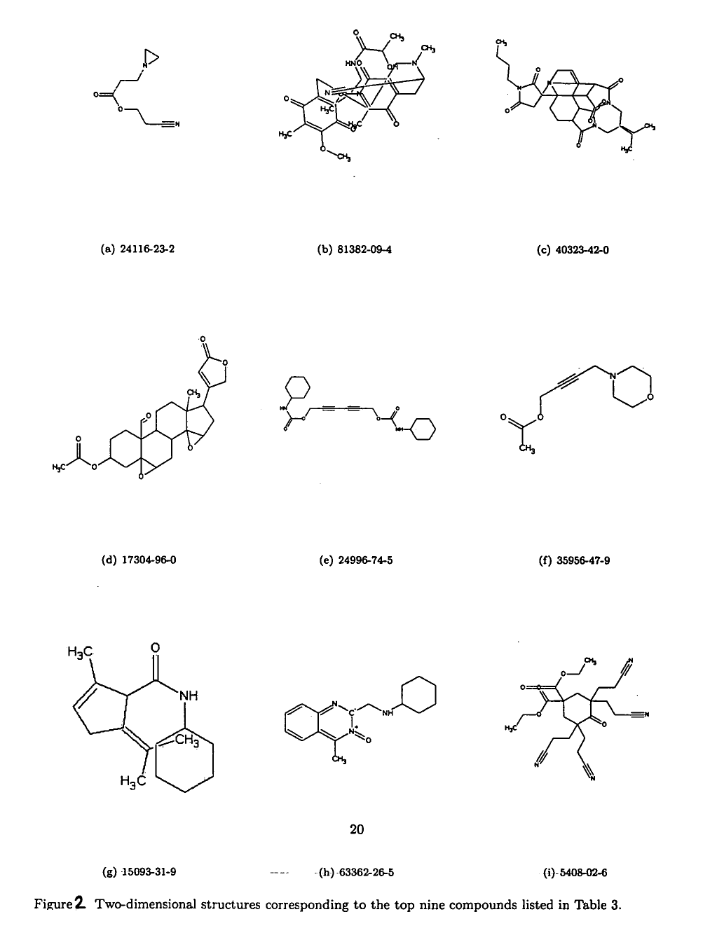

### DETAILED DESCRIPTION AND PREFERRED EMBODIMENTS

The invention is a method of representing biopolymers in a computational trainable system for use in the prediction of the interaction of proteins with other proteins, nucleic acids, small molecules and biopolymers. The interactions are determined in a pairwise fashion, with higher order structures containing more than two components being determined in multiple rounds of analysis. A collection of known biomolecular interactions, such as protein-protein interactions, are encoded as a set of features on a residue-by-residue basis in the trainable system. Databases of heterogenous protein-protein interactions exist, including the publicly-accessible Database of Interacting Proteins (DIP: http://dip.doe-mbi.ucla.edu) which at the time of this application contains 10933 interaction pairs. Other databases contain information regarding protein interactions in single organisms; one such database is available at http://pim.hybrigenics.com, which contains all of the known protein-protein interactions known in the bacteria H. pylori The selection of a database is not a limiting aspect of the invention. Moreover, the databases listed should not be considered static entities or to be limited to the data that they contain at the time of the application. The databases are a source of training sets to “teach” the trainable system, but are not a component of the invention itself. The invention is instead the manner in which the biopolymers are represented as a linear set of features and used in the trainable system to predict the interactions of the encoded biopolymers with other molecules.

The accuracy of the predictive model is dependent upon the quality of the database used. The more the system is “taught” in the number of biomolecular interactions entered into the database, and the greater the similarities between the molecules to be compared, the higher the predictive value of the model will be. Alternatively, limiting the members of the query group to a single cell compartment (e.g. endoplasmic reticulum, nucleus, Golgi apparatus) increases the accuracy of the predictive model by eliminating possible interactions between proteins that would never come into contact with each other in the context of the cell.

A trainable system is defined as a program, algorithm or other analytical method into which data are input in the form of a training set from which the system can “learn” to determine patterns and that will allow for predictions of outcomes, upon analysis of unknowns similar to those in the training set. “Learning” and analysis of the unknown samples may be performed by any of a number of methods including the use of a support vector machine (SVM), neural network, classification and regression analysis (CART), Bayesian networks, or other algorithms, software programs or a combination thereof. In the instant invention the training set is a group of pairs of biomolecules that do or do not interact that are used to “teach” the system what characteristic features do or do not interact such that the unknowns can be analyzed for the presence of features such that interactions may be predicted. The training set may be augmented or modified and should not be considered a static entity. The invention is not limited by the algorithm, software or hardware used, but instead is dependent on the method used to train the system such that predictions on interactions can be made based on linear sequence information or primary structure of biomolecules, rather than based on tertiary structure.

A training set is defined as a collection of data, typically derived from a database, containing examples of pairs of biomolecules that do or do not interact. The examples of biomolecular interaction or non-interaction are analyzed by a trainable system so it may “learn” how classes of biomolecules interact. The type of biomolecular interactions to be determined (e.g. protein-protein, protein-nucleic acid) in the group of biomolecules with unknown interactions would determine the selection of the type of training set. The training set may be augmented or modified during the process of analysis.

A biomolecule is defined as a protein, peptide, nucleic acid, complex lipid or carbohydrate, small molecule such as a growth factor, hormone, vitamin, lipid, carbohydrate, neurotransmitter, signalling molecule, amino acid or nucleotide, a scaffold for attachment of cells, a polymer for the use in the assembly of organ, joint or other implant, a bioactive agent such as a drug.

Primary or linear structure is defined as the sequence of nucleotides or amino acids in the nucleic acid or polypeptide of interest, respectively. The primary structure of a biomolecule is defined as a representation of orgainc or inorganicinorganic molecules as a sequence of constituentconstituent elements.

For example, in the invention, a training set “teaches” the trainable system about biomolecular interactions by providing examples of how proteins interact with each other by providing a number of examples of protein-protein interactions. Proteins in the query group are matched to the proteins in the database based on homology. Proteins in the query group are predicted to interact based on the interactions of their homologs in the database. For example, if protein A in the database is homologous to protein A′ of the query group either in a portion or along the entire length of the protein, and protein B in the database is homologous to protein B′ in the query group, and proteins A and B are known to interact, proteins A′ and B′ are predicted to interact. As interactions tend to take place through modular domains in the protein (e.g. SH2 and SH3 domains, zinc fingers, leucine zippers, amphipathic helicies), predictions may be made accurately even if the proteins in the query group do not have overall high homology to proteins in the database. However, the greater similarity of the organisms in the query and database groups, the better the prediction accuracy of the method.

The invention is a method for whole-proteome interaction mapping wherein, the database comprises all of the experimentally-known or hypothesized protein-protein interactions of a single organism. Protein sequences comprising a partial or complete proteome from a different organism, that may or may not contain any defined protein-protein interaction, are analyzed by the trainable system for homology between proteins in the database and the query group. Homologous proteins of interacting pairs in the database are predicted to interact with each other. Proteins are analyzed on an all-against-all basis with each potential pairwise combination being analyzed. The learning machine may be used for subsequent rounds of analysis to predict higher order structures containing greater than two proteins.

Data obtained through use of the trainable system can be tested in a laboratory setting to confirm interactions. Such data can be entered into the system for subsequent rounds of analysis and to further “teach” the system about additional protein-protein interactions. As more data are entered into the system, the predictive ability of the system increases.

The invention is a method for the use of a trainable system to predict the presence of epitopes of interest, including functional domains and binding sites of proteins, and antigenic determinants. By casting the numerical optimization procedure as a regression problem, a continuous value for binding affinity of ligand-molecular complex can be learned. In this manner the same scheme for representing linear biopolymer sequences as features is used, and the training procedure involves “sliding” a window along the query sequence, each step outputting a numerical value that constitutes a predicted interaction value of the sequence within the window and the query ligand. Example public-domain databases containing data appropriate for training the system in this mode are: (1) The Ligand Chemical Database for Enzyme Reactions (http://www.genome.ad.jp/dbget/ligand.html), (2) The Function Immunology Database of MHC molecules, antigens and diseases (FIMM; http://sdmc.krdl.org.sg:8080/fimm/), and (3) the ImMunoGeneTics database (IMGT; http://www.ebi.ac.uk/imgt/).

The invention is a method for the use of a trainable system to predict the binding of nucleic acids with proteins. This mode of prediction is carried out similarly to the antigenic determinant prediction scheme outlined above. Training data for local interactions between nucleic acid molecules (DNA, or RNA) and proteins are developed from the nucleic acid-protein complex structural data of the Protein Data Bank (PDB; http://www.rcsb.org/pdb/) and summarized in the DNA-Protein Interaction Database (DNAPIDB; http://www.dpidb.belozersky.msu. ru/). The sites of interaction are analyzed as before and converted to a set of features in the learning machine. The trained system outputs a thresholded-score indicative of the local propensity for nucleic acid binding at each site along the query protein.

The invention is a method for predicting biochemical, signal transduction and gene regulatory circuit pathways in the cell, using information obtained from the use of various modes of the trainable system to predict small molecule-protein, protein-protein, and protein-nucleic acid interaction pairs. Proteins analyzed by the trainable system may be subdivided based on cell compartment. Protein-protein interactions have been experimentally demonstrated using proteins that would never interact due to compartmentalization within cells. Proteins can be divided into groups based on cellular compartmentalization for entry into the trainable system for analysis (e.g. endoplasmic reticulum and Golgi apparatus for glycosylation machinery; nuclear proteins for DNA repair factors). Pathways may also be subdivided by the location of various processes in the cell. Signal transduction pathways involve the binding of small molecules by cell surface receptors (e.g. epidermal growth factor receptor, large G-protein receptors), followed by transmission of a signal via a number of cytosolic factors, some of which shuttle in and out of the nucleus, (e.g. kinases, adaptor proteins) to transcription factors in the nucleus (e.g. fos and jun). Thus, one can limit the potential interactions that can be determined by the use of the invention by limiting the input query to proteins that would have the opportunity to interact in the cell.

The invention is a method for cell-map proteomics. Biochemical, signaling and gene regulatory path ways can be mapped for entire organisms. The entire genome of the Helicobacter pylori, which contains coding sequences for 486 proteins, has been sequenced and 1,039 protein-protein interactions have been mapped. Using this model organism, which performs all of the functions required for viability, one can map the interactions of genomes of similar organisms, such as Campylobacter jejuni, an enteric bacteria pathogen that causes common symptoms of food poisoning. A complete protein-protein interaction map for C. jejuni computed using the methods disclosed herein is available at http://www-bioeng.ucsd.edu/cjbean/. Analysis of the major constituent protein domains shows a high degree of similarity. These orthologous bacterial proteomes represent a model system for demonstrating the utility of the invention for performing proteome wide interaction mining. The accuracy of the proteome map will depend on the quality of the database as well as the level of similarity of the organisms to be analyzed. The higher the similarity and the greater the number of interactions defined, the greater the predictive value of the information in the database.

### EXAMPLE 1

Databases of known biomolecular interactions. Databases of protein interactions are available at multiple sites including the Database of Interacting Proteins (DIP) http://dip.doe-mbi.ucla.edu which currently contains 10933 entries, and the H. pylori database, http://pim.hybrigenics.com which contains 1273 interacting pairs between the 486 potential proteins of the organism. In the DIP database, each interaction pair contains fields representing accession codes for other pubic protein databases, protein name identification and references to experimental literature underlying the interacting residue ranges, and protein-protein complex dissociation constants. The protein interaction domain coverage within the DIP is diverse; at least 175 distinct domains are represented. The proteins are predominantly eukaryotic, with a majority of the proteins being from the yeast Saccharomyces cerevisiae. The information in the database is updated constantly by individuals studying protein-protein interactions, thus providing an increasing number of interactions that may be “taught” to the trainable system of the invention.

A summary of public domain databases containing data appropriate for training this invention are listed in the following table. The entries in this table represent only a small subset of currently-available databases, which continue to appear and grow in size.

TABLE 1
Databases Useful for Training Systems Described in this Invention

| Database | Type of | | |
|---|---|---|---|
| Name | Data | Size | URL |
| Database of | Assorted | 10,933 | http://dip.dombi. |
| Interacting | protein- | interactions | ucla.edu |
| Proteins (DIP) | protein | | |
| | interactions | | |
| Protein | Whole- | 1273 protein- | http://pim. |
| Interaction Map | proteome | protein | hybrigenics.com |
| (PIM) | protein- | Interactions | |
| | protein | | |
| | interactions | | |
| | for H. pylori | | |
| Biomolecular | Protein- | 5939 | http://www. |
| Interaction | protein | interactions, | bind.ca/ |
| Network DB | interactions, | 54 | |
| (BIND) | molecular | complexes, 7 | |
| | complexes | pathways | |
| | and | | |
| | pathways | | |
| MIPS | Genetic, | Total | http://mips.gsf.de/ |
| Saccharomyces | physical | statistics | proj/yeast/CYGD/ |
| cerevisiae- | interactions | unlisted | db/index.html |
| Interaction | in yeast | | |
| Tables | | | |
| Functional | functional | 400 protein | http://sdmc.krdl. |
| IMMunology | immunology, | antigens, 1200 | org.sg:8080/fimm/ |
| Database | focusing on | peptides, 800 | |
| (FIMM) | MHC, | HLA | |
| | antigens, | sequences, 50 | |
| | and disease | diseases | |
| SYFPEITHI | | Total | http://syfpeithi.bmi- |
| | | statistics | heidelberg.com/ |
| | | unlisted | Scripts/MHCServer. |
| | | | dll/Info.html#head |
| Drosophila | Drosophila | Total | http://cmmg.biosci. |
| Protein | protein | statistics | wayne.edu/finlab/ |
| Interaction Map | interactions | unlisted | PIMdb.htm |
| Database | | | |
| DNA-Protein | 3D | Total | http://www.dpidb. |
| lnteraction | structures of | statistics | belozersky.msu.ru/ |
| Database | complexes | unlisted | |
| (DNAPIDB) | in which | | |
| | protein | | |
| | binds either | | |
| | DNA or RNA | | |

### EXAMPLE 2

Support vector machine (SVM) learning. The protein-protein interaction estimator can utilize the technique of “support vector” learning, an area of statistical learning theory subject to extensive recent research (Vapnic, 1995; Schökopf et al., 1999). The trainable system algorithm is not a limiting aspect of the invention. The method described in this invention can be used in conjunction with any exemplar-based machine learning paradigm, including, for example, neural networks, classification and regression trees (CART), or Bayesian networks. While in principle any of these or other learning algorithms would work with this invention, it is believed that SVM represents the best machine learning method for this invention, for the following reasons:
1. SVM generates a representation of the nonlinear mapping from biopolymer sequence to protein fold space using relatively few adjustable model parameters.
2. Based on the principle of structural risk minimization, SVM provides a principled means to estimate generalization performance via an analytic upper bound on the generalization error.
3. SVM is characterized by fast training, which is essential for high-throughput screening of large biological databases

The trainable system can be trained to classify labeled empirical data points by constructing an optimal high-dimensional decision function that (1) maximizes the separations between classes and (2) minimizes the “structural risk”
R( )=□Q(z,)dF(z)\tilde{,}□
with respect to perameters using an independently, identically distributed (i.i.d.) sample Z=(z₁, z₂, . . . zᵢ) generated by an (unknown), underlying probability distribution F, where Q is an indicator function, and □ is a set of parameters. Sample points zᵢ=(xᵢ, yᵢ) comprise protein features \tilde{x}ᵢ□ⁿ and their classifications \tilde{y}ᵢ{−1, 1}. In practice, the learning task converges rapidly as a constrained quadratic program is solved. The resultant decision function h represents an hypothesis generator for interference on novel data points, mapping them onto the discrete set y, or h:x□y. This is a binary decision (+1 □ interaction, −1 □ no interaction).

### EXAMPLE 3

Feature representation. For each amino acid sequence of a protein-protein complex, feature vectors were assembled from encoded representations of tabulated residue properties (Ratner et al., 1996) including charge, hydrophobicity and surface tension for each residue in the sequence. This set of features is not a limiting aspect of the invention. Instead any set of physical, chemical or biological features corresponding in a discrete or spatially-averaged sense to each residue or nucleotide in a linear biopolymer sequence may be used to construct an example for training the system described in this invention. These features are then concatenated to create an interaction pair example. Negative examples (i.e. putative non-interacting pairs) were generated by randomly extracting individual proteins from the database and randomizing their amino acid sequence while preserving their chemical composition. This randomization technique is well established for statistical significance estimation in biological sequence analysis.

### EXAMPLE 4

Analysis of protein-protein interactions using the DIP database. DIP database samples were at random, and data were partitioned into training and testing sets, at approximately a 1:1 ratio. Feature vectors were constructed in this manner and were used as examples for training and testing the learning machine. Testing examples were not exposed to the system during SVM learning. The database is robust in the sense that it represents a compendium of protein interaction data collected from diverse experiments. At least 175 protein domains are represented. There is a negligible probability that the learning system will “learn its own input” on a narrow, highly self-similar set of data examples. This enhances the generalization potential of the trained support vector machine.

Software methods for parsing the DIP database, control of randomization and sampling of records and sequences, and feature vector creation were developed in Java. A new database was constructed by augmenting the original DIP records. Additional fields added included amino acid sequence data and associated residue features as described in Example 3.

Support Vector Machine learning was implemented using Joachims' SVM^{light} (Joachims, 1999), available online at http://www-ai.cs.uni-dortmund.de/SOFTWARE/SVM_LIGHT.eng.html.

Training and testing exemplar data files were developed using maximum allowed residue length as an input parameter to the data preparation software. This threshold length was used to selectively filter out certain protein interactions from consideration as means to explore possible residue length dependence of the generalization accuracy of the SVM. A different SVM was trained for each maximum residue length threshold case. Residue length thresholds of of 350, 500, 750, 1000 and □ in the numerical experiments were considered.

The performance of each SVM was evaluated using the inductive accuracy of on the previously unseen samples as a metric. “Inductive accuracy” is defined here as the percentage of correct protein interaction predictions in the test set, including positive and negative interaction examples.

The main result of the protein-protein interaction predictions are summarized in the system generalization accuracy summary in Table 2. “Inductive accuracy” is the percentage of correct protein interaction predictions on test data not previously seen by the system. Each row in the table corresponds to a fixed residue length threshold used to generate the training and testing examples. Data in the column marked “# Examples” indicate the total number of training and testing examples for each case. During data preparation, at the shortest residue length thresholds, the random sampling procedure ignores database records more frequently as the threshold test is violated; this results in greater disparity between the train/test data counts.

| | # Examples | Inductive |
|---|---|---|
| Residue Cutoff | (train, test) | Accuracy |
| 350 | (122, 172) | 51.33% |
| 500 | (448, 380) | 67.37% |
| 750 | (1020, 1094) | 65.63% |
| 1000 | (1616, 1648) | 68.63% |
| □ | (2218, 2240) | 70.40% |

The data demonstrate that as the volume of available training data increases, nearly two out of three potential protein interactions are correctly estimated by the system. When all of the data are included, the inductive accuracy reaches 70.4%. Apparently, even though the marginal contribution to the total protein interaction density function is very slight when including the longest protein in the analysis, these additional data points assist the SVM with the description of the margin. This observation is consistent with the nature of SVMs as. margin classifiers, where a few key data examples near the decision boundary are sufficient to specify the boundary between the classes.

### EXAMPLE 5

Analysis of protein-nucleic acid interactions. The invention can be, used to predict the binding of nucleic acids with proteins. This mode of prediction is carried out by casting the numerical optimization procedure as a regression problem. A continuous value for binding affinity of DNA/RNA-protein complex can be learned. In this manner the same scheme for representing linear biopolymer sequences as features is used, and the training procedure involves “sliding” a window along the query sequence, each step outputting a numerical value that constitutes a predicted interaction value of the sequence within the window and the query ligand.

Training data for local interactions between nucleic acid molecules (DNA, or RNA) and protiens can be developed from the nucleic acid-protein complex structural data of the Protein Data Bank (PDB; http://www.rcsb.org/pdb/) and summarized in the DNA-Protein Interaction Database (DNAPIDB; http://www.dpidb.belozersky.msu.ru/). The sites of interaction are analyzed as before and converted to a set of features in the learning machine. The trained system outputs a thresholded-score indicative of the local propensity for nucleic acid binding at each site along the query protein.

### EXAMPLE 6

Prediction of protein epitopes. The invention is a method for the use of a learning machine to predict the presence of epitopes of interest, including functional domains and binding sites of proteins, and antigenic determinants. The learning algorithm in this application is cast as a regression similarly to the DNA/RNA-protein determinant prediction scheme outlined above._Example public-domain databases containing data appropriate for training the system in this mode are: (1) The Ligand Chemical Database for Enzyme Reactions (http://www.genome.ad.jp/dbget/ligand.html), (2) The Function Immunology Database of MHC molecules, antigens and diseases (FIMM; http://sdmc.krdl.org.sg:8080/fimm/), and (3) the ImMunoGeneTics database (IMGT; http://www.ebi.ac.uk/imgt/).

### EXAMPLE 7

Whole proteome interaction analysis. The invention may be applied to larger scale studies of protein-protein interactions in a proteome wide scale. Application of a “phylogenetic bootstrap” method for protein-protein interaction mining, which comprises traversal of a phenogram, interleaving rounds of computation and experiment, to develop a knowledge base of protein interactions in genetically similar organisms. The steps comprising the phylogenetic bootstrap are distilled into an algorithm, described herein in detail.

The algorithm.

Input: Proteome sequences sₐ, s_{b}, labels Yₐ.

Input: Parameters., ε_{cv}^{max}

Assume: similarity □ (F(Zₐ), F(Z_{b}))≦.

Compute: feature set Xₐ, sample Zₐ

1. Xₐ□ get Features (sₐ)
2. Zₐ⁺□{(x, y)x□ Xₐ, y□ Yₐ, y=+1}
3. Zₐ⁻□{(x, y)x□ Xₐ, y□ Yₐ, y=−1}
4. Zₐ□ Zₐ^{+U} Zₐ⁻
   Compute: decision rule on sample
5. h(,x)□SVM (Zₐ)
   Compute: C.V. generalization error estimate
6. ε_{cv}□ L00({h})
7. Prob{\hat{_{y}}=y. h}~1−ε_{cv}
   Assert: ε_{cv}^{v}≦ε_{cv} ?
   Compute: feature set X_{b}
8. X_{b}□ get Features (S_{b})
   Compute: predict interactions
9. \hat{_{Y}}b □h.(, X_{b})
   Assert: validate sample experimentally
10. Z_{b}□{(x, \hat{_{y}}). x□ X_{b}, \hat{_{y}}□ _{Ŷ}
   Assert: ε_{cv}^{v}≦ε_{cv}?
   Input: New proteome sequences s_{c}
   Update: sₐ, s_{b}, labels Yₐ
11. sₐ □ sₐ+s_{b}; Yₐ□ Yₐ+\hat{_{Y}}b; s_{b}□ s_{c}
   Goto: Step 1; iterate while ε_{cv}^{v}≦ε_{cv}≦ε_{cv}^{max}

The phylogenetic bootstrap algorithm above is summarized in this section. A procedural step identified by the pattern “S[num]” refers to Step #[num] in the accompanying Box entitled “Phylogenetic bootstrap algorithm“.

Input: First, it is necessary to specify the species Sₐ, S_{b} subject to investigation. In general, some existing protein interaction data may be at hand for each proteome, although their relative cardinality may be quite skewed, as discussed above. Our line of thought assumes that no interaction data are available for S_{b}; we have only a set of labels {Yₐ} corresponding to experimentally-verified interactions sampled from the proteome of species Sₐ. These labels, along with the amino acid sequence sets {sₐ} and {s_{b}} comprising the species' respective proteomes, are inputs to the algorithm.

Other inputs required are the inter-proteome distance (Eq. 2), and the maximum allowable rate of generalization error, ε_{cv}^{max}, where 0≦ε_{cv}^{max}<0.5.

S1-S4: Construct features based on attributes of the primary structure sequences {sₐ} from the training dataset. Encoded attributes Xₐ for entire proteomes may be derived from tabulated residue properties including charge, hydrophobicity, and surface tension as described previously (Bock and Gough, 2001). At this stage, data preprocessing including normalization and filtering should be performed to produce a useful sampled attribute set {x.x ε□ⁿ, ⊂ X}. A total of /data points z are constructed by adding labels y to the accepted feature vectors x, or zᵢ=(xᵢ, yᵢ), i=1, . . . ,/. The union of positively- and negatively-labeled examples constitutes the training sample {Zₐ}.

S5: Design an optimal support vector machine to classify data points in the sample {Zₐ}. After learning, the system builds a decision rule h that maps data vectors xᵢ onto the classification space yᵢε[−1,1]. The numerical sign of yᵢ is interpreted as the likelihood that the two proteins represented by xᵢ will interact.

S6-S7: Perform leave-one-out cross-validation experiments on the training set. For each observation zᵢ, train an SVM using all other points {z.zεZₐ, z□zᵢ}., and predict the class membership of the omitted point zᵢ. Accumulate the total number of misclassifications observed in this process. Take the final average cross-validation error as the estimated generalization error rate ε_{cv} of the learner h.

S8: Construct features X_{b} from sequences {s_{b}} for the unlabeled proteome S_{b}. All-vs-all pairwise interactions may be represented in the prediction set. The same data preparation process should be applied as in S1.

S9: Predict a new set of protein-protein interactions {Ŷ_{b}} via the trained system; h( ): x_{b}□ Ŷ_{b}, where are parameters of the model. To the extent that the assumption of proteomic similarity □ (F(Zₐ), F(Z_{b}))≦. is satisfied, each point estimate ŷ is expected to be accurate with a probability g( )(1−ε_{cv}), or Prob {ŷ=y. \tilde{h} g( )(1−ε_{cv}).

S10: Take a random sample from the protein interaction prediction set Z_{b}={(x, ŷ).x ⊂ X_{b}, ŷ ⊂ Y_{b}) and verify the predicted protein interactions (both positive and negative) using experimental proteomics techniques. Compare the experimentally-validated and calculated estimated prediction error rates. Assert that the following statement holds true: where the ε_{cv}^{v}≦ε_{cv}≦ε_{cv}^{max} superscript “v” denotes validate by experiment.

Input: Select sequences {s_{c}} from a new, related organism S_{c}. The similarity assumption □ (F(Zₐ), F(Z_{c}))≦. must still be maintained.

S11: Add sequences from the validated prediction set to the training set, and consider this expanded set as the training set for the next iteration: {sₐ}={sₐ}+{s_{b}}. Update the class labels by adding the prediction label set {Yₐ}={Yₐ}+{Ŷ_{b}}. Protein interactions for organism Sc will now be computed.

Return to S1 and repeat the process.

The stopping condition for this iteration is violation at any time of the assertions regarding the generalization error rate, i.e. when the error rate from LOO, ε_{cv} exceeds the specified limit ε_{cv}^{max}, or when the experimental observations contain more frequent errors than the calculated rate, or ε_{cv}^{v}>ε_{cv}.

Assumptions

The support vector machine (Vapnik, 2000) can be trained to classify labeled empirical data points by constructing an optimal high-dimensional decision function that (1) maximizes the separation between classes and (2) the minimizes “structural risk”
R( )=□Q(z, )dF(z), ~□ (1)
with respect to parameters . using an independently, identically-distributed (i.i.d) sample Z=(zᵢ, z₂, . . . zᵢ) generated by an (unknown) underlying probability distribution F, where Q is an indicator function, and □ is a set of parameters. Sample points zᵢ=(xᵢ, yᵢ) comprise protein features xᵢε□ⁿ and their classifications yᵢε{−1, 1}. In practice, the learning task converges rapidly as a constrained quadratic programming is solved. The resultant decision function h represents an hypothesis generator for inference on novel data points, mapping them onto the discrete set y, or h:x□y. This is a binary decision (+1→interaction, −1→no interaction). The assumption of a fixed generative probability distribution F(Z) in Eq. 1 is a key issue in the design of the data mining application. A consequence of this assumption is that a system trained on a sample Zₐ, taken from species Sₐ, may be used to predict interactions on a sample Z_{b} from another species S_{b}, provided that features of their respective protoeomes are not too dissimilar in some sense, or
□(F(Zₐ),F(Z_{b}))≦. (2)
where ρ is a distance metric and is a small positive constant. The statistic ρ is general, and may signify cross-species similarity based on genome-level “edit distance” (Sanko. et al. 1992), whole-proteomic content (Tekaia et al. 1999), or molecular structures (Woese et al. 1990), to cite only three of many possibilities.

Interaction mining analysis as embodied in the phylogenetic bootstrap algorithm detailed above makes certain assumptions about the distributions of proteomic data in the design sample Z. Other assumptions inherent in this approach include:
1. Static intracellular state. If proteins A and B interact in species S1, they will also interact if co-occurring in species S2. This assumption may not be generally valid for different physiological conditions present in S2 relative to S1.
2. Completeness of design sample. Any pair of proteins (A,B) not labeled as interactors in the design sample Z are assumed to not interact. This is a subtle but significant point that must be held in mind when interpreting prediction results.
3. Proximity. The all-vs.-all computational screen selects interaction pairs based on primary structure, and does not discriminate protein subcellular location. Such analysis could be done in a separate post-mining filtering step.
4. Simple interactions. Only binary interactions are represented; complexes of proteins with more than two components are only inferred indirectly in post-mining analysis. This further implies that modifications to protein A (e.g., phosphorylation, glycosylation) prerequisite to its recognition by B are not identified.

### EXAMPLE 8

### Virtual Screen for Ligands of Orphan G-protein Coupled Receptors

Members of the superfamily of G protein-coupled receptors (GPCRs) are among the most widely-screened classes of signal transduction targets, due to their intrinsic association with disease-related signalling pathways and track record of therapeutic success. These receptors have undergone intense historical scrutiny as potential drug targets. The structural and functional diversity of GPCRs continue to present opportunities to develop novel drugs.

It is estimated that approximately 160 GPCR-encoding genes in the human genome have yet to be functionally characterized by sequence homology, or through association with known endogenous ligands. These are called orphan GPCRs (oGPCRs), which bind unknown ligands. The physiological role of oGPCRs can only be elucidated by first identifying cognate peptides or small molecule ligands that modulate their function.

This example presents a virtual screening methodology that generated a ranked list of high-binding small molecule ligands for oGPCRs, circumventing the requirement for receptor three-dimensional structure determination. Features representing the receptor are based only on physicochemical properties of primary amino acid sequence, and ligand features use the two-dimensional atomic connection topology and atomic properties.

The experimental screen comprised nearly 2 million hypothetical oGPCR-ligand complexes, from which we observed that the top 1.96% predicted affinity scores corresponded to highly active” ligands against orphan receptors. Results representing predicted high-scoring novel ligands for many oGPCRs are presented here.

This virtual screening approach is used in support of the functional characterization of oGPCRs by identifying potential cognate ligands. This approach finds use for identifying leads and active agents of pharmaceutical therapies to modulate the activity of faulty or disease-related cellular signaling pathways. In addition to application to cell surface receptors, this approach is a generalized strategy for discovery of small molecules that may bind intracellular enzymes and involve protein-protein interactions.

Cell signal transduction is a regulatoary mechanism that connects a stimulation or binding event at the cell surface with its consequent intracellular physiological effect. An important superfamily of cell surface receptors which implement this signal transduction paradigm are the G protein-coupled receptors (GPCRs), so-named for their mediation of intracellular heterotrimeric G proteins (2). The molecular mechanisms underlying the modulation of GPCR-stimulated signaling, and the connection to other cellular signaling pathways may be quite elaborate (3). Defective signaling in cells is often closely linked to disease (4). Dysfunctional GPCR-mediated signal transduction systems in particular have been shown to play a role in a number of pathological states, including endocrine diseases (5), cancer (6), retinitis pigmentosa (7), nephrogenic diabetes insipidus (8), neurological or psychiatric disorders (9), asthma and rhinitis (10), and cardiac disease (11).

Analysis of the human genomic sequence suggests there may be 750 human GPCR-encoding genes, of which approximately 160 cannot be functionally characterized either on the basis of sequence homology or by association with known endogenous ligands (19). These are referred to as orphan GPCRs receptors (oGPCRs) which bind (as yet) unknown ligands (20, 21). The physiological role of oGPCRs can only be elucidated by first identifying cognate peptides or small molecule ligands which modulate their function. Afterwards, a significant task remains specifically to establish bioactivity in the face of non-specific GPCR ligand binding, and to isolate pathway associations of the ligand binding event given complex second messenger responses (22). This present invention involves a step in a method to determine the physiological role of GPCRs and oGPCRs and identifying bioactivity of ligands identified by this method. A step in the method involves discriminating small molecule ligands for oGPCRs using the screening approach of the present invention.

Experimental ligand identification strategies in the art have been based upon reverse pharmacology” (23), in which an oGPCR is cloned and expressed in a cell line, then transfected into tissue extract containing endogenous ligands presumed to bind the receptor with high affinity. Finally, biological and pharmaceutical activity and association of the ligands to pathological states is assessed (24). Previous investigators have proposed structure-based virtual screens for ligands, which can be categorized as ligand-based or receptor-based methods (reviewed in (25). The ligand-based methods extrapolate from properties of compounds (“pharmacophores”) known to bind a target receptor, by searching databases for compounds with similar properties.

In the present invention, a different approach was taken. It is assumed for this invention that high-affinity ligands are unknown. The receptor-based methods use computational docking procedures to bind compounds from a ligand database to the binding site of the receptor of interest. This presupposes that the three-dimensional structure of the receptor is available. For GPCRs, such an approach has limited utility; integral membrane proteins continue to be difficult to crystallize, constraining the analysis to a small number of structurally known GPCRs (26).

The approach of the present invention involves bioinformatics methods. The method virtually screens for ligands of orphan G-protein coupled receptors. This method is based on a machine learning approach which estimates the binding free energy between a small-molecule ligand and a receptor protein (29). A distinct advantage of this approach is the simplicity of requisite input data: proteins are described using only physicochemical properties of primary amino acid sequence, and ligand features are based on the two-dimensional connectivity between constituent atoms and atomic properties. In application, large numbers of chemical compounds may be screened against a particular oGPCR sequence, with a ranked list of putative high-affinity ligands generated automatically on output.

This screening approach functional characterizatizes oGPCRs by identifying potential cognate ligands, thereby identifying strategies to direct the therapeutic regulation of important signaling pathways in the cell.

Quantitative Receptor Pharmacology

GPCRs are important regulators of central nervous system function in health and disease (30). Accordingly, in this example, a data set of known psychoactive drugs and their associated ligand binding affinities was used to create a discriminative statistical model of ligand-receptor interaction.

The data examples used in this example were derived from the PDSP Ki Database, a public repository containing information on affinities between real or candidate drugs and GPCRs and other receptors found in the central nervous system (33) http://pdsp.cwru.edu.

Ligand-receptor affinties used to generate this data set were estimated using a variety of experimental protocols, many of which are described in detail on the PDSP web site. Data collected during binding assays can be compared across protocols and laboratories by expressing the results in terms of a normalized index of affinity (or, reciprocally, dissociation) for a given ligand-receptor complex. One such expression in common usage is given by the Cheng-Prusoff equation (34) for competitive radioligand binding, given by

$${K}_{i} = {IC}_{50} * ( 1 + \frac{[ {L}^{*} ]}{{K}_{d}} ) \qquad ( 1 )$$

where Ki is the equilibrium dissociation constant for the analyte of interest ([L]), IC₅₀ is the concentration of ligand displacing 50% of the specifically bound labeled ligand [L*], and Kd is the (inverse) affinity of the radioligand for the receptor. Ki represents the equilibrium concentration of unlabeled ligand that would bind half the receptor binding sites in the absence of radioligand or other competitors. A fundamental pharmacological characteristic of the receptor-drug complex, Ki may be used as the basis for evaluating different candidate drugs. Inference of biological activity for a single compound can be made based on the computed value
pKi=−In(Ki) (2)

To assign degree of bioactivity to pKi, this investigation followed the convention listed in Table 3 (35). Table 3 shows a relationship between negative logarithm of the dissociation constant (pKi) and biological activity. This scheme may be used to infer biological activity of a single ligand-receptor complex, or to rank order a library of compounds bound to a receptor in experimental screening. (See GPCRDB—reference 35). Values pKi>7 are generally taken to imply high binding affinity.

Alternatively, qualitative comparisons between elements of a group of compounds are possible by their rank-ordering in terms of binding affinity for a given receptor (e.g., see (36)). The supposition is that the highest-affinity ligands are correlated with efficacy of pharmacological effect, either as agonists or antagonists, which is the approach taken in the present investigation, where we predict and rank the values of pKi for a large number of drug-like, small molecule ligands in the specific context of a set of orphan G protein-coupled receptors.

Support Vector Regression

The support vector machine (SVM) is a pattern recognition algorithm that may be used for regression estimation (31, 32) is described above.

$$f ( x ) = {\sum }_{i = 1}^{t} ( {α}_{i}^{*} - {α}_{i} ) k ( {x}_{i} , x ) + b \qquad ( 3 )$$

where xεR^{d} are observations, a* and aᵢ are Lagrange multipliers of the constrained quadratic optimization problem, k is a kernel function measuring the similarity between its arguments, b is the intercept, and l is the number of example data pairs. Usually only a subset of the coefficients aᵢ*, aᵢ are nonzero; the associated observations xᵢ are called the support vectors, and their sparsity contributes to the efficient computation of the expansion in Equation 3, while providing an analytic upper bound on the generalization error (37).

The function approximation is constructed based on an i.i.d. sample Z
Z={z₁, . . . , zᵢ}={(xᵢ, yᵢ), . . . , (x₁, y₁)}, zεR^{d}×R (4)
where yᵢεR is the target value corresponding to training vector xᵢ. The kernel function k maps patterns x from “input space” to a higher-dimensional “feature space” [symbol] via a nonlinear map
Φ:R^{d}→εR^{D} (5)
(where in general, D>>d) and constructs a linear regression in this high(possibly infinite)-dimensional space. In (38) it was noted that because Φ enters the optimization problem as inner products, finding an expression for inner products in feature space [symbol] in terms of input data points x would obviate the requirement to discover and explicitly compute the feature map Φ. This provides for computational tractability. For example, in the case of a Gaussian kernel
k(x₁, x₂)=exp(−||x₁−x₂||²/(2σ²)) (6)
F bas infinite dimension, however an SVM regression may be readily computed to estimate the function f(x) within this feature space. The consequence is that nonlinear, high dimensional “mixing” of individual components of x₁, x₂ in feature space may elicit subtle patterns contributing to an effective regression. The regression experiments reported here were carried out using the LIBSVM software package (39).
Virtual Screening Approach

In this example, the support vector regression algorithm was used to approximate the unknown function f(x) which connects descriptors of known receptor-ligand pairs to their experimentally-determined dissociation constants pKi.

This function was then evaluated using data patterns corresponding to uncharacterized oGPCR-ligand pairs, producing predicted values for pKi. These predictions were sorted, producing a ranked list of chemical compounds most likely to bind to the orphan receptor.

In this approach, no three-dimensional structural information on either the receptor or small molecule ligand was required to construct an accurate nonparametric regression function.

Preparation of Example Data

Descriptive features. Numerical descriptor arrays (“feature vectors” x in Equation 3) representing attributes distinguishing ligand-receptor complexes were derived using the procedure described in (29).

Target receptors. Target features comprised numerical values for surface tension, isoelectric point and accessible surface area attributed each amino acid comprising the receptor primary structure. Tables of residue physicochemical properties are widely accessible; one source of such data is The Amino Acid Repository at http://www.imb-jena/IMAGE AA.html.

This scheme encodes physicochemical properties of the primary structure that are likely to influence the thermodynamics of binding. Next, this vector of floating point numbers is transformed (by interpolation or decimation) onto a fixed-length sequence, an essential step to maintain a consistent physical\meaning” for each transformed vector element across examples.

Chemical ligands. Ligand features were established using a two-dimensional molecular connectivity map to exemplify the arrangement of each compound's constituent atoms in space. Each ligand's 2-D molecular connection array was supplemented by numerical values for essential chemical properties of the component atoms, including ionization potential, electron affinity and density. The rationale followed again was to propose quantities relating to the physics of binding. All two-dimensional arrays of numbers were assembled into a row matrix, projected onto one dimension using the singular value decomposition (41), and finally resampled to yield a fixed-length sequence representing each small-molecule compound.

Target-ligand complexes. Individual receptor-ligand feature vectors designed in this manner were finally concatenated to produce feature vectors for training and testing the support vector regression machine.

Training source database. To construct training examples, target-ligand complexes were selected from the PDSP Ki database. From the nominal Ki database comprising over 26; 000 records, a useable subset of 9,075 complexes was identified, based on the ability to associate amino acid sequences with receptors, and SMILES strings (42) with their cognate ligands, respectively. Statistical redundancy between training examples in any supervised learning situation may result in unreliable cross-validated estimates of generalization error. To address this issue, highly-similar examples were excluded within the training data set according to the following procedure:
1. A similarity matrix SεR^{ial} was created for the l=9,075 ligand-target complexes found within PDSP. Each matrix element s_{i,j} expresses the degree of similarity between example feature vectors numbered i and j. Values s_{i,j} were evaluated using an heuristic criterion:

$${s}_{i , j} = \frac{1}{d} {\sum }_{k = 1}^{d} {H}_{B} ( \vert {x}_{i , k} - {x}_{j , k} \vert \leq σ ) , 0 < {s}_{i , j} \leq 1 \qquad ( 7 )$$
where H_{B} is Heaviside's step function with Boolean argument, σεR^{d} is the standard deviation, estimate for each attribute, and k denotes a feature. In essence, this equation counts the number of corresponding vector elements in xᵢ and xⱼ whose values differ by less than one standard error.

2. Redundant examples were removed, referring to the similarity matrix, using a two-pass algorithm designed for this application. The idea is to eliminate training examples based upon their composite pattern and label similarities.
(a) The first pass iterates over each row i of S, evaluating the similarity of training vector xᵢ to all other vectors {xⱼ}, j=1, . . . , ↓, j≠i. Those examples where the similarity to xᵢ exceeds a numerical threshold criterion are marked for removal subject to subsequent passes of the algorithm. This investigation used a threshold value 0,98.
(b) For each data vector xᵢ, the second pass compares its target value yᵢ to each value {yⱼ}, j=1, . . . , ↓, j≠i associated with examples marked as “similar” in the previous pass. The target quantities to be learned by the regression represent binding affinity (pKᵢ); where pKᵢ between respective training instance differed by less than 0.25 logarithm units, the redundant example was excluded from further analysis.

This process removed 3,756 redundant observations (41%), leaving a total of 5,310 examples for cross-validation training from the pre-redundancy processed set. The median target value pKi in this set was μ=6.32, with extreme values ranging between −9.8 and +11.

Testing source database. Testing examples, forming the basis for the prediction of binding affinities for novel oGPCR complexes, were generated using (i) orphan G protein-coupled receptor sequences found within the Swiss-Prot Protein Knowledgebase (43), and (ii) a “druglike” subset of compounds derived from the National Cancer Institute (NCI) open databases as provided within the Ligand.Info Small-Molecule Databases (44) (downloadable at http://liqand.info/.

An alternative approach to drug design using this method involves a process which filters non-druggable compounds before beginning biological receptor activity screening (45). From the 69,045 druglike compounds stored in Ligand.Info, 34,753 were selected based on the availability of an unique CAS registry number or NSC accession ID.

The nominal list of orphan receptors (data found in file “7tmlist.txt” dated June 2, 2004. This list may be accessed at http://www.expasv.org/cgi-bin/lists?7tmrlist.txt.) contained 135 targets. Many of the orphan receptors represented nearly identical amino acid sequences from different organisms. We analyzed this set of sequences using global, multiple sequence alignment implemented in the program DBClustal(46), with an E-value cutoff of 10.⁻⁴⁰ This E-value was previously used to analyze evolutionary relationships within families of GPCRs (47).

The global alignment produced clusters of sequentially-similar receptors; from each, a single archetypical receptor was selected. The resulting set of oGPCRs consisted of 55 targets, for which putative cognate ligands would be identified. These orphan receptors, including their cluster sizes, are summarized in Table 4.

Feature vectors were built by connecting the 55 oGPCRs with the 34,753 druglike chemical compounds in our locally-constructed database using the methods described above. The resulting set of feature vectors encoding hypothetical oGPCR-ligand complexes (n=1,911,415) was processed using the trained support vector regression function of Equation 3 to estimate values for their binding affinities.

Standardization of examples. Attributes were mean-corrected and standardized by considering all training and testing data vectors simultaneously as a single matrix of observations. Overall mean and sample standard deviation statistics were calculated for each column (feature) of this matrix; these in turn became normalizing factors that were applied to all data examples.

Experimental Procedure

The PDSP-derived training examples were used to develop an optimal support vector regressor. A number of schemes have been proposed in the literature to systematically select support vector machine model parameters; e.g., see (48, 49, 50). The approach followed here searched a computational grid of parameters of the learning machine, identifying the best parameter set using 10-fold cross-validation. Let us denote target-compound affinity scores using the variable y to simplify notation, or
y:=pKi (8)

Each held-out data partition was evaluated by computing the normalized mean squared error (NMSE)

$$NMSE = \frac{{\sum }_{i = 1}^{{l}_{p}} {( {y}_{i} - {\hat{y}}_{i} )}^{2}}{{\sum }_{i = 1}^{{l}_{p}} {( {y}_{i} - \overline{y} )}^{2}} \qquad ( 9 )$$

where yᵢ is a predicted value for yᵢ, \overline{y} is the true mean, and l_{F} is the number of compounds in the prediction set. Equation 9 shows that an observed value of NMSE=1 corresponds to simply predicting the mean value of the dependent variable (51); values less than 1 imply predictive value-added by a particular method.
NMSE is related to the coefficient of determination by (R²) by R²≈1−NMSE, suggesting that NMSE be interpreted as a coefficient of non-determination—a measure of the percentage of variance in y that is not explained by the model.

The support vector regression model exhibiting the lowest overall NMSE was selected for the ensuing virtual screen. The optimal model used a Gaussian kernel (Equation 6), with parameter values C=10 (train error/margin tradeoff), γ=0.01 (inverse kernel width), and v=0:5 (solution sparsity) (32, 37) .

Model selection. Average statistics over the ensemble of cross-validation folds may provide an error estimate of the generalization performance of the regression model (52). The selected model produced a cross-validated prediction error NMSEcv=0.57. This corresponds to a predictive Rcv=0.43. The standard and maximum deviation between actual and predicted pKi were SD=1.21 and MD=8.12, respectively.

The central tendency of the population of predicted affinity scores corresponded to a “weakly active” binding affinity according to the calibration protocol of Table 3. Our interest lies within the “highly active” region [insert y-hat]>7; predicted affinity scores lying in this region constituted 1.96% (37,407/1,911,415) of all results in our numerical experiments. The methodology therefore screened out 98% of the putative oGPCR-ligand complexes.

A total 4,357 different compounds were represented within the set of high-affinity ligands. This translates to. about 12% of the complete set of drug-like compounds comprising the virtual screen.

Cross-target Analysis of High-affinity Ligands

Many of the drug-like ligands were predicted to bind strongly to more than a single target oGPCR. This observation suggested the possibility of analyzing structural characteristics of these compounds, providing insights into recurring motifs or pharmacophores. Such information would also aid in the design of bioactive compounds for families of receptors based on so-called molecular fingerprints (55).

We performed cross-target analysis by calculating the average binding score for each ligand across the set of target receptors. Bioactivity for compounds with the highest average scores was assessed according to functional information found in the online NCI chemical structure database (Located at this URL: http://cactus.cit.nih.gov/ncidb2.)

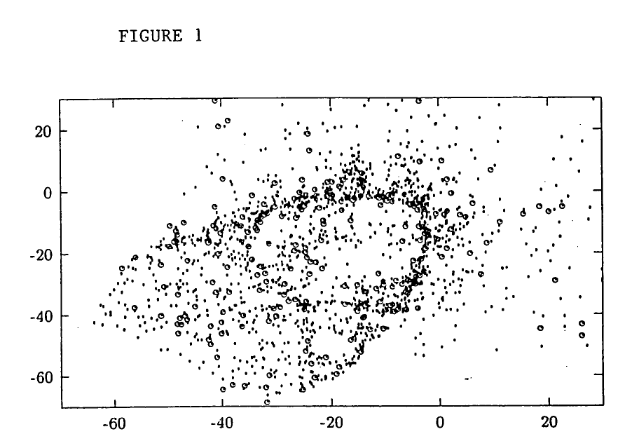

For a given compound, the log-odds ratio that it is associated with biological function F is

$$LOR = ln ( \frac{p ( F )}{p ( ∼ F )} ) \qquad ( 11 )$$

where p(F) is the probability that F is present, and p(˜F) is the probability it is not. LOR values indicate the probability that the quantity in the numerator evaluates to “true”; confidence increases with the magnitude of LOR. A listing of 13 compounds with the strongest generalized binding affinity is presented in Table 5. The table includes values for average pKi, biological activities with some relevance to GPCRs or GPCR-modulated functions, and the associated LOR. FIG. 1 illustrates two-dimensional structures corresponding to the top nine compounds listed in Table 5.

Activities represent independent predictions made using the program PASS, which computes probabilities based on structure-activity relationships (56). As an example, the first compound listed in Table 5 is associated with four different putative biological activities. Each of these functions are connected to G protein-coupled receptor-related conditions or processes reported in the literature: vasodilation (57), rhinitis (10), mediator release (58) and histamine activation (59). This suggests that the cross-target binding ligands found in the present virtual screen are plausible in the GPCR context, according to bioactivities attributed to these compounds by independent, structure-based calculations. These are highly specific predictions, given the range of possible activities.

Top Binding Compounds for oGPCRs

The main results of this research are summarized in Tables 6-7, which present the highest-scoring ligands for oGPCRs produced by the virtual screen. Target receptors are identified by number and Swiss-Prot accession,to provide cross-reference to their definition in Table 4. The columns marked “#y>7” list the number of binding affinity scores predicted to be “highly active” for the corresponding receptor. Parenthetically, this number is shown as a fraction of all scores computed for the corresponding receptor sequence. We have chosen to present only the top 3 scoring compounds for each oGPCR (shown in column “CAS No.”), due to space constraints.

It is readily seen that this methodology is selective, filtering out all but a very small percentage of the ligand-target complexes presented to the support vector machine. For all orphan G protein-coupled receptors considered, the number of high-scoring virtual “hits” varies from 0 (23:6% or 13/55 cases studied) to 3,958 (Receptor #16; Swiss-Prot protein Q14330 (60)). A large majority of the top-binding ligands for all of the orphan receptor targets included one or both compounds with CAS registry numbers 24116-32-2 or 81382-094. These two ligands were identified in Table 5 as being the most highly cross-reactive, and this is reflected in their frequent appearance in Tables 6-7. The first small molecule compound, CAS #24116-32-2, is known by the chemical name 2-cyanoethyl 3-(1-aziridyinyl)propanoate, but little information on its pharmaceutical applications is available in public databases (outside of the NCI database). The second compound, CAS81382-09-4, is a relatively large (mol. wt. 564.6 g/mol), DNA-binding antibiotic, and appears to have strong antitumor properties. This compound is known commonly as “Saframycin A”. Many more strong-affinity ligands were predicted for over 75% of the oGPCRs; their exact numbers can be found in the tables.

We have chosen to screen orphan receptors mainly from human tissue (c.f. Table 4). A great many oGPCR sequences, however, appear to be highly conserved across species. This study clustered the target sequences to reduce the analysis set such that a single representative from each sequence-based cluster was used. Where a small number of ligands are predicted to bind a particular, conserved target, a method of the invention involves steps of employing high-throughput experimental screening techniques, and obtaining empirical binding data of that target against a complete set of specific ligands as predicted here. The objective would be to ascertain the degree of biological relevance of the predictions. The method thereby involves identifying mechanisms of mediation of important signalling pathways.

This example demonstrated a virtual screening methodology that circumvented the requirement for receptor three-dimensional structure determination. The invention is used to directly generate a ranked list of high-binding small molecule ligands for oGPCRs. An advantage of this approach is the simplicity of the requisite input data: proteins are described using only physicochemical properties of primary amino acid sequence, and ligand features are based on the two- dimensional connectivity between constituent atoms, and their chemical properties. This virtual screening approach is used in support of the functional characterization of oGPCRs by identifying potential cognate ligands. The method predicts ligand binding energy at a given receptor.

The support vector machine approach described here is deterministic in the sense that the trained regression function will produce a consistent output for each ligand-target complex, without appealing to three-dimensional pose or difficult statistical mechanics calculations. The experimental screen comprised more than 1.9 million hypothetical oGPCR-ligand complexes, from which it was observed that less than 2% of predicted affinity scores corresponded to “highly active” ligands against orphan receptors. In practice, different numerical thresholds or data scaling procedures may be applied to further reduce the set of putative oGCPR ligands under consideration.

The method of the invention provides a ranked list of conjectured ligand-oGPCR complexes, which find use in methods to validate them by experimental ligand binding assays. Further methods which use the findings of these assays involve assays to identify bioactivity of the ligands and receptors and ultimate association to cellular pathways and cascaded second messenger responses (22). The method of the invention achieves these objectives in the development of for pharmaceutical therapies to modulate or short-circuit faulty or disease-related cellular signaling pathways.

The methodology described here is general, and may be applied to other receptor types. Two non-limiting embodiments of applications of therapeutic importance include design of tyrosine kinase inhibitors (64) or nuclear receptors. In the latter, the present method is useful in the design of hormone analogs to bind defective receptors. One only requires access to the amino acid sequence of the modified receptor; the procedures reported here could be easily adapted to provide a sensitive means to investigate small variations in the properties of a ligand (which may be a peptide, for example) (65).

ln addition to cell surface receptors, this approach is a generalized strategy for discovery of small molecules which may bind intracellular enzymes and involve protein-protein interactions. Small-molecule mediated inhibition of protein-protein interactions is considered to be the most difficult of these drug design objectives, in part owing to the discrepancy in physical size between small molecule and the targeted protein complex (66). This approach provides a method of addressing this problem.

### References Cited in Example 8

[1] M Rodbell. Signal transduction: Evolution of an idea. Bioscience Reports, 15(3):117{133, June 1995.

[2] A G Gilman. G proteins: Transducers of receptor-generated signals. Annual Review of Biochemistry, 56:615{649, July 1987.

[3] U Gether. Uncovering molecular mechanisms involved in activation of G protein-coupled receptors. Endocrine Reviews, 21(1):90{113, February 2000.

[4] T Hunter. Signaling{2000 and beyond. Cell, 100(1):113{127, Jan. 7 2000.

[5] Z Farfel, H R Bourne, and T liri. The expanding spectrum of G protein diseases. New England Journal of Medicine, 340(13):1012{1020, Apr. 1 1999.

[6] J S Gutkind. Cell growth control by G protein-coupled receptors: From signal transduction to signal integration. Oncogene, 17(11 Reviews):1331{1342, Sep. 17, 1998.

[7] S T Menon, M Han, and T P Sakmar. Rhodopsin: Structural basis of molecular physiology. Physiological Reviews, 81 (4):1659{1688, October 2001.

[8] A M Spiegel. Inborn errors of signal transduction: mutations in G proteins and G protein-coupled receptors as a cause of disease. Journal of Inheritable Metabolic Diseases, 20(2):113{121, June 1997.

[9] M Rocheville, D C Lange, U Kumar, S C Patel, R C Patel, and Y C Patel. Receptors for dopamine and somatostatin: Formation of hetero-oligomers with enhanced functional activity. Science, 288(5463): 154{157, Apr. 7 2000.

[10] E N Johnson and K M Druey. Heterotrimeric G protein signaling: Role in asthma and allergic inflammation. Journal of Allergy and Clinical Immunology, 109(4):592{602, April 2002.

[11] J T Meij. Regulation of G protein function: Implications for heart disease. Molecular and Cellular Biochemistry Journal, 157(1-2):31{38, Apr. 12-26 1996.

[12] D S Auld, D Diller, and K-K Ho. Targeting signal transduction with large combinatorial collections. Drug Discovery Today, 7(24):1206{1213, Dec. 15 2002.

[13] G Muller. Towards 3D structures of G protein-coupled receptors: a multidisciplinary approach. Current Medicinal Chemistry, 7(9):861{888, September 2000.

[14] F Gasparini, R Kuhn, and J-P Pin. Allosteric modulators of group I metabotropic glutamate receptors: novel subtype-selective ligands and therapeutic perspectives. Current Opinion in Pharmacology, 2(1): 43{49, 2002.

[15] A D Howard, G McAllister, S D Feighner, Q Liu, R P Nargund, L Van der Ploeg, and A A Patchett. Orphan G-protein coupled receptors and natural ligand discovery. Trends in Pharmacological Sciences, 22(3):132{140, March 2001.

[16] P Ma and R Zemmel. Value of novelty? Nature Reviews Drug Discovery, 1(8):571{572, August 2002.

[17] H E Hamm. The many faces of G protein signaling. Journal of Biological Chemistry, 273(2):669{672, Jan. 9 1998.

[18] T-H Ji, M Grossmann, and I Ji. G protein-coupled receptors. I. Diversity of receptor-ligand interactions. Journal of Biological Chemistry, 273(28):17299{172302, Jul. 10 1998.

[19] A Wise, K Gearing, and S Rees. Target validation of G-protein coupled receptors. Drug Discovery Today, 7(4):235{246, Feb. 15 2002.

[20] O Civelli, H P Nothacker, Y Saito, Z Wang, S H Lin, and R K Reinscheid. Novel neurotransmitters as natural ligands of orphan G-protein-coupled receptors. Trends in Neurosciences, 24(4):230{237, April, 2001.

[21] D-S Im. Orphan G protein-coupled receptors and beyond. Japanese Journal of Pharmacology, 90(2): 101{106, (2002).

[22] O Civelli. Functional genomics: the search for novel neurotransmitters and neuropeptides. FEBS Letters, 430(1-2):55{58, Jun. 23 1998.

[23] F Libert, G Vassart, and M Parmentier. Current developments in G-protein-coupled receptors. Current Opinion in Cell Biology, 3(2):218{223, April 1991.

[24] S Wilson, D J Bergsma, J K Chambers, A l Muir, K G Fantom, C Ellis, P R Murdock, N C Herrity, and J M Stadel. Orphan G-protein-coupled receptors: the next generation of drug targets? British Journal of Pharmacology, 125(7):1387{1392, December 1998.

[25] P D Lyne. Structure-based virtual screening: an overview. Drug Discovery Today, 7(20):1047{1055, Oct. 15 2002.

[26] J Ballesteros and K Palczewski. G protein-coupled receptor drug discovery: Implications from the crystal structure of rhodopsin. Current Opinion in Drug Discovery & Development, 4(5):561{574, September 2001.

[27] G Milligan. Strategies to identify ligands for orphan G-protein-coupled receptors. Biochemical Society Transactions, 30(4):789{793, August 2002.

[28] H Kubinyi. The design of combinatorial libraries. Drug Discovery Today, 7(9):503{504, May 1 2002.

[29] J R Bock and D A Gough. A new method to estimate ligand-receptor energetics. Molecular and Cellular Proteomics, 1:904{910, November 2002.

[30] M N Pangalos, C Davies, and C H Davies. Understanding G Protein-Coupled Receptors & Their Role in the CNS. Oxford University Press, Oxford, UK, January 2003.

[31] V N Vapnik. The Nature of Statistical Learning Theory. Springer-Verlag, Heidelberg, Germany, 1995.

[32] B SchÄolkopf, P Bartlett, A J Smola, and R Williamson. Support vector regression with automatic accuracy control. In L Niklasson, M Boden, and T Ziemke, editors, Proceedings of the Eighth International Conference on Artificial Neural Networks, pages 111{116, 1998.

[33] B L Roth, E Lopez, S Patel, and W K Kroeze. The multiplicity of serotonin receptors: Uselessly diverse molecules or an embarrassment of riches? The Neuroscientist, 6(4):252{262, August 2000.

[34] Y Cheng and W H Pruso®. Relationship between the inhibition constant (K1) and the concentration of inhibitor which causes 50 per cent inhibition (I50) of an enzymatic reaction. Biochemical Pharmacology, 22(23):3099{3108, Dec. 1 1973.

[35] F Horn, E Bettler, L Oliveira, F Campagne, F E Cohen, and G Vriend. GPCRDB information system for G protein-coupled receptors. Nucleic Acids Research, 31(1):294{297, Jan. 1 2003.

[36] M J Millan, M Brocco, J-Michel Rivet, V Audinot, A Newman-Tancredi, L Maio⁻ss, S Queriaux, N Despaux, J-L Peglion, and A Dekeyne. S18327 (1-[2-[4-(6-°uoro-1, 2-benzisoxazol-3-yl)piperid-1-yl]ethyl]3-phenyl imidazolin-2-one), a novel, potential antipsychotic displaying marked antagonist properties at alpha(1)- and alpha(2)-adrenergic receptors: II. Functional pro⁻le and a multiparametric comparison with haloperidol, clozapine, and 11 other antipsychotic agents. Journal of Pharmacology and Experimental Therapeutics, 292(1):54{66, January 2000.

[37] B SchÄolkopf, A J Smola, R Williamson, and P Bartlett. New support vector algorithms. Neural Computation, 12:1083{1121, 2000.

[38] B E Boser, I M Guyon, and V N Vapnik. A training algorithm for optimal margin classi⁻ers. In D Haussler, editor, Proceedings of the Fifth Annual ACM Workshop on Computational Learning Theory, pages 144{152, Pittsburgh, Pa., 1992. ACM Press.

[39] C-C Chang and C-J Lin. Training [symbol] support vector classir⁻ers: Theory and algorithms. Neural Computation, 13(9):2119{2147, 2001.

[40] J R Bock. Biomolecular Interactions Using Machine Learning. PhD thesis, Department of B.ioengineering, University of California San Diego, 2003.

[41] G H Golub and C F van Loan. Matrix Computations. Johns Hopkins University Press, Baltimore, Md., 2nd edition, 1989.

[42] D Weininger. SMILES, a chemical language and information system. 1. Introduction to methodology and encoding rules. Journal of Chemical Information and Computer Sciences, 28(1):31{36, 1988.

[43] B Boeckmann, A Bairoch, R Apweiler, M-C Blatter, A Estreicher, E Gasteiger, M J Martin, K Michoud, C O'Donovan, I Phan, S Pilbout, and M Schneider. The SWISS-PROT protein knowledgebase and its supplement TrEMBL in 2003. Nucleic Acids Research, 31(1l):365{370, Jan.1 2003.

[44] M von Grotthuss, J Pas, and L Rychlewski. Ligand-Info, searching for similar small compounds using index pro⁻les. Bioinformatics, 19(8):1041{1042, May 22 2003.

[45] C A Lipinski. Drug-like properties and the causes of poor solubility and poor permeability. Journal of Pharmacological and Toxicological Methods, 44(1):235{249, 2000.

[46] J D Thompson, F Plewniak, J Thierry, and O Poch. DbClustal: rapid and reliable global multiple alignments of protein sequences detected by database searches. Nucleic Acids Research, 28(15):2919{2926, Aug. 1 2000.

[47] R C Graul and W Sadee. Evolutionary relationships among G protein-coupled receptors using a clustered database approach. AAPS Pharm Sci, 3(2):E12, May 4 2001.

[48] O Chapelle and V Vapnik. Model selection for support vector machines. In S A Solla, T K Leen, and K-R Muller, editors, Advances in Neural Information Processing Systems 12, Cambridge, Mass., 2000. MIT Press.

[49] S S Keerthi and C-J Lin. Asymptotic behaviors of support vector machines with Gaussian kernel. Neural Computation, 15(7):1667{1689, July 2003.

[50] A Chalimourda, B SchÄolkopf, and A Smola. Experimentally optimal [symbol] n support vector regression for di®erent noise models and parameter settings. Neural Networks, 17(1):127{141, 2004.

[51] N A Gershenfeld and A S Weigend. The future of time series: Learning and understanding, volume XV of Sante Fe Institute Studies in the Sciences of Complexity, pages 1{70. Addison-Wesley, Reading, Mass., 1993.

[52] J K Martin and D S Hirschberg. Small sample statistics for classi⁻cation error rates I: Error rate measurements. Technical Report ICS-TR-96-21, Department of Information and Computer Science, University of California Irvine, Jul. 2 1996.

[53] H Gohlke, M Hendlich, and G Klebe. Predicting binding modes, binding a±nities and “hot spots” for protein-ligand complexes using a knowledge-based scoring function. Perspectives in Drug Discovery and Design, 20:115{144, 2000.

[54] W T Eadie, D Drijard, F E James, M Roos, and B Sadoulet. Statistical Methods in Experimental Physics. North-Holland, Amsterdam, Netherlands, 1971.

[55] D C Greenbaum, W D Arnold, F Lu, L Hayrapetian, A Baruch, J Krumrine, S Toba, K Chehade, D Bromme, I D Kuntz, and M Bogyo. Small molecule a±nity ⁻ngerprinting: a tool for enzyme family subclassir⁻cation, target identir⁻cation, and inhibitor design. Chemistry & Biology, 9(10):1085{1094, October 2002.

[56] V V Poroikov, D A Filimonov, W D Ihlenfeldt, T A Gloriozova, A A Lagunin, Y Borodina, A V Step-anchikova, and M C Nicklaus. PASS biological activity spectrum predictions in the enhanced open NCI database browser. Journal of Chemical Information and Computer Sciences, 43(1):228{236, January-February 2003.

[57] A D Eckhart, T Ozaki, H Tevaearai, H A Rockman, and W J Koch. Vascular-targeted overexpression of G protein-coupled receptor kinase-2 in transgenic mice attenuates beta-adrenergic receptor signaling and increases resting blood pressure. Molecular Pharmacology, 61 (4):749{758, April 2002.

[58] H Ali, J Ahamed, C H-Munain, J L Baron, M S Krangel, and D D Patel. Chemokine production by G-protein-coupled receptor activation in a human mast cell line: Roles of extracellular signal-regulated kinase and NFAT. The Journal of Immunology, 165:7215{7223, 2000.

[59] G Bertaccini and G Coruzzi. An update on histamine H3 receptors and gastrointestinal functions. Digestive Diseases and Sciences, 40(9):2052{2063, September 1995.

[60] I Gantz, A Muraoka, Y K Yang, L C Samuelson, E M Zimmerman, H Cook, and T Yamada. Cloning and chromosomal localization of a gene (GPR18) encoding a novel seven transmembrane receptor highly expressed in spleen and testis. Genomics, 42(3):462{4662, Jun. 15 1997.

[61] L C James and D S Taw⁻k. The speci⁻city of cross-reactivity: Promiscuous antibody binding involves speci⁻c hydrogen bonds rather than nonspeci⁻c hydrophobic stickiness. Protein Science, 12(10):2183{2193, October 2003.

[62] C Bissantz, G Folkers, and D Rognan. Protein-based virtual screening of chemical databases. 1. Evaluation of di®erent docking/scoring combinations. Journal of Mediciinal Chemistry, 43(25):4759{4767, Dec. 14 2000.

[63] R V Rebois, B G Allend, and T E Hebert. The targetable G protein proteome: Where is the next generation of drug targets? Drug Discovery Today: Targets, 3(3), June 104-111 2004.

[64] J R Woolfrey and G S Weston. The use of computational methods in the discovery and design of kinase inhibitors. Current Pharmaceutical Design, 8(17):1527{1545, 2002.

[65] M Habeck. New ligands for defective receptors. Drug Discovery Today, 8(6):236{237, Mar. 15 2003.

[66] T R Gadek and J B Nicholas. Small molecule antagonists of proteins. Biochemical Pharmacology, 65(1): 1{8, Jan. 1 2003.

### Other References

Champion, M. M. et al. (2001) Functional native-state proteomics in E. coli. In Proceedings of Proteomics: From Proteins to Drugs. San Francisco, Calif., Jun. 21-22, 2001. Cambridge Healthtech Institute.

Enright, A. J. et al. (1999) Protein interaction maps for complete genomes based on gene fusion events. Nature 402:86-90.

Fields, S. and O.-K. Song (1989) A novel genetic system to detect protein-protein interactions. Nature 340:245-6.

Joachims, T. (1999) Making Large-Scale Support Vector Machine Learning Practical. In Advances in Kernel Mehods—Support Vecotr Learning, ch. 11, pp. 169-84, MIT Press, Cambridge, Mass.

MacBeath, G. and S. L. Schreiber (2000) Putting proteins as microarrays for high throughput funciton determination. Science 289:1760-3.

Ratner, B. D. et al. (1996) Biomaterials Science: An Introduction to materials in Medicine, Academic Press, San Diego, Calif. 1996.

Sankoff, D. et al. (1992) Gene order comparisons of phylogenetic interference: Evolution of the mitochondrial genome. Proc. Natl. Acad. Sci. USA 89: 6575-9.

Schölkopf, B. et al. (1999) Advances in Kernel Methods: Support Vector Learning, MIT Press, Cambridge, Mass., 1999.

Tekia, F. et al. (1999) The genomic tree as revealed from a whole proteome comparisons. Genome Res. 9:550-7.

Vapnik, V. (1995) The Nature of Statistical Learning Theory. Springer-Verlag, New York, N.Y.

Woese, C. R. et al. (1990) Towards a natural system of organisms: Proposal for the domains Archea, Bacteria, and Eucarya. Proc. Natl. Acad. Sci. USA 87:4576-4579.

Although exemplary embodiments of the invention are described above by way of example only, it will be understood by those skilled in the field that modifications may be made to the disclosed embodiment without departing from the scope of the invention, which is defined by the appended claims.

TABLE 3
Relationship between negative logarithm of the dissociation
constant (pKᵢ) and biological activity. This scheme may be
used to infer biological activity of a single ligand-receptor
complex, or to rank order a library of compounds bound
to a receptor in experimental screening. Source: GPCRDB (35).

| pKᵢ | Inferred Activity |
|---|---|
| >7 | highly active |
| 6-7 | active |
| 5-6 | weakly active |
| <5 | inactive |

TABLE 4
Orphan G protein-coupled receptors used in the virtual screen. The objective
is to find ligands which bind strongly to these receptors, without
knowledge of receptor structure in three-dimensional space.
oGPCRs taken from the file 7tmrlist.txt dated 2-Jun-2004.

| | Swiss-Prot | Swiss-Prot | | | Clust. |
|---|---|---|---|---|---|
| No. | name | accession | Description | Species | size |
| 1 | MAS_HUMAN | P04201 | Mas proto-oncogene | H. sapiens | 3 |
| 2 | MRS_HUMAN | P35410 | Mas-related MRS (MAS-R) | H. sapiens | 4 |
| 3 | CML1_HUMAN | Q99788 | Chemokine receptor-like 1 | H. sapiens | 3 |
| 4 | CML2_HUMAN | Q99527 | Chemokine receptor-like 2 | H. sapiens | 2 |
| 5 | EBI2_HUMAN | P32249 | EBV-induced GPCR 2 | H. sapiens | 2 |
| 6 | ETB2_HUMAN | O60883 | Endothelin B receptor-like | H. sapiens | 2 |
| 7 | H963_HUMAN | O14626 | Probable GPCR | H. sapiens | 6 |
| 8 | LGR4_HUMAN | Q9BXB1 | Leucine-rich GPCR 4 | H. sapiens | 8 |
| 9 | RDC1_HUMAN | P25106 | GPCR RDC1 homolog | H. sapiens | 4 |
| 10 | GP61_HUMAN | Q9BZJ8 | Probable GPCR | H. sapiens | 2 |
| 11 | GPR1_HUMAN | P46091 | Probable GPCR | H. sapiens | 7 |
| 12 | GPR3_HUMAN | P46089 | Probable GPCR | H. sapiens | 7 |
| 13 | GPR4_HUMAN | P46093 | Probable GPCR | H. sapiens | 2 |
| 14 | GP10_HUMAN | P49683 | Probable GPCR | H. sapiens | 5 |
| 15 | GP15_HUMAN | P49685 | Probable GPCR | H. sapiens | 7 |
| 16 | GP18_HUMAN | Q14330 | Probable GPCR | H. sapiens | 1 |
| 17 | GP19_HUMAN | Q15760 | Probable GPCR | H. sapiens | 3 |
| 18 | GP20_HUMAN | Q99678 | Probable GPCR | H. sapiens | 1 |
| 19 | GP21_HUMAN | Q99679 | Probable GPCR | H. sapiens | 1 |
| 20 | GP22_HUMAN | Q99680 | Probable GPCR | H. sapiens | 1 |
| 21 | GP26_HUMAN | Q8NDV2 | Probable GPCR | H. sapiens | 4 |
| 22 | GP27_HUMAN | Q9NS67 | Probable GPCR | H. sapiens | 10 |
| 23 | GP31_HUMAN | O00270 | Probable GPCR | H. sapiens | 4 |
| 24 | GP32_HUMAN | O75388 | Probable GPCR | H. sapiens | 1 |
| 25 | GP33_MOUSE | O88416 | Probable GPCR | M. musculus | 2 |
| 26 | GP34_HUMAN | Q9UPC5 | Probable GPCR | H. sapiens | 3 |
| 27 | GP35_HUMAN | Q9HC97 | Probable GPCR | H. sapiens | 2 |
| 28 | GP39_HUMAN | O43194 | Probable GPCR | H. sapiens | 1 |
| 29 | GP40_HUMAN | O14842 | Probable GPCR | H. sapiens | 1 |
| 30 | GP41_HUMAN | O14843 | Probable GPCR | H. sapiens | 3 |
| 31 | GP45_HUMAN | Q9Y5Y3 | Probable GPCR | H. sapiens | 4 |
| 32 | GP52_HUMAN | Q9Y2T5 | Probable GPCR | H. sapiens | 1 |
| 33 | GP57_HUMAN | Q9P1P4 | Probable GPCR | H. sapiens | 2 |
| 34 | GP62_HUMAN | Q9BZJ7 | Probable GPCR | H. sapiens | 1 |
| 35 | GP80_HUMAN | Q96P68 | Probable GPCR | H. sapiens | 3 |
| 36 | GP82_HUMAN | Q96P67 | Probable GPCR | H. sapiens | 1 |
| 37 | GP92_HUMAN | Q9H1C0 | Probable GPCR | H. sapiens | 1 |
| 38 | G101_HUMAN | Q96P66 | Probable GPCR | H. sapiens | 2 |
| 39 | G151_HUMAN | Q8TDV0 | Probable GPCR | H. sapiens | 3 |
| 40 | G152_HUMAN | Q8TDT2 | Probable GPCR | H. sapiens | 2 |
| 41 | G160_HUMAN | Q9UJ42 | Probable GPCR | H. sapiens | 1 |
| 42 | G161_HUMAN | Q8N6U8 | Probable GPCR | H. sapiens | 1 |
| 43 | GRE1_BALAM | Q93126 | Probable GPCR | B. amphitrite | 3 |
| 44 | YWO1_CAEEL | Q10904 | Probable GPCR | C. elegans | 1 |
| 45 | YWO4_CAEEL | Q10907 | Probable GPCR | C. elegans | 1 |
| 46 | YS96_CAEEL | Q09965 | Putative GPCR | C. elegans | 2 |
| 47 | YS97_CAEEL | Q09966 | Putative GPCR | C. elegans | 1 |
| 48 | YT66_CAEEL | Q11082 | Probable GPCR | C. elegans | 1 |
| 49 | YKR5_CAEEL | P34311 | Probable GPCR | C. elegans | 2 |
| 50 | YLD1_CAEEL | Q03566 | Probable GPCR | C. elegans | 1 |
| 51 | YY13_CAEEL | Q18775 | Probable GPCR | C. elegans | 1 |
| 52 | YYO1_CAEEL | Q18904 | Probable GPCR | C. elegans | 1 |
| 53 | YMJC_CAEEL | P34488 | Putative GPCR | C. elegans | 1 |
| 54 | YR13_CAEEL | Q09638 | Probable GPCR | C. elegans | 1 |
| 55 | YN84_CAEEL | Q03613 | Probable GPCR | C. elegans | 1 |

TABLE 5
Compounds with high cross-target affinity. CAS No. is the CAS
registry identifier. ŷ is the average predicted value of pKᵢ,
taken over at least one receptor. Activities and log-odds ratios
(LOR) are adapted from the NCI open database. These results
suggest that the predicted cross-target binding ligands are
plausible in the GPCR context, according to bioactivities attributed
to these compounds by independent, structure-based calculations.

| CAS No. | ŷ (avg.) | Putative Activities | LOR |
|---|---|---|---|
| 24116-23-2 | 7.59 | Vasodilator | 4.06 |
| | | Rhinitis treatment | 3.45 |
| | | Mediator release inhibitor | 3.18 |
| | | Histamine release stimulant | 2.92 |
| 81382-09-4 | 7.52 | Histamine release stimulant | 1.68 |
| 40323-42-0 | 7.46 | Cardiovascular analeptic | 3.09 |
| | | Hypnotic | 2.58 |
| | | Alzheimer's treatment | 1.94 |
| 17304-96-0 | 7.45 | Analeptic | 4.80 |
| | | Respiratory analeptic | 4.56 |
| | | Spasmogenic | 3.35 |
| | | Antidyskinetic | 3.35 |
| 24996-74-5 | 7.44 | CNS muscle relaxant | 3.85 |
| | | Sedative | 3.79 |
| | | Anticonvulsant | 3.08 |
| | | Muscle relaxant | 3.05 |
| 35956-47-9 | 7.44 | Cholinergic agonist | 5.10 |
| | | Acetylcholine agonist | 4.90 |
| | | Acetylcholine muscarinic agonist | 4.56 |
| | | Acetylcholine antagonist | 3.65 |
| 15093-31-9 | 7.44 | Cholinergic agonist | 2.25 |
| | | Neurological disorders treatment | 2.13 |
| | | Nootropic | 1.76 |
| 63362-26-5 | 7.44 | Bronchodilator | 4.58 |
| | | Neurological disorders treatment | 2.06 |
| | | Analgesic | 1.71 |
| 5408-02-6 | 7.43 | Prostaglandin antagonist | 4.60 |
| | | Spasmolytic | 3.11 |
| | | Acetylcholine release stimulant | 3.06 |
| | | Acetylcholine muscarinic antagonist | 3.00 |
| 79005-55-3 | 7.43 | Cognitive disorders treatment | 3.18 |
| | | Alzheimer's treatment | 2.83 |
| | | Vasodilator | 2.11 |
| | | Antipruritic | 1.87 |
| 35878-52-5 | 7.42 | Antidyskinetic | 2.18 |
| | | Cardiovascular analeptic | 1.35 |
| 15569-50-3 | 7.42 | Cardiovascular analeptic | 4.60 |
| | | Parathyroid hormone antagonist | 3.50 |
| 6630-45-1 | 7.41 | Cannabinoid receptor agonist | 4.22 |
| | | Neurotrophic factor | 3.69 |
| | | Cardiovascular analeptic | 3.60 |

TABLE 6
oGPCRs and Predicted High-Affinity Ligands. Targets are
identified by number and Swiss-Prot accession, providing
cross-reference to Table 4 Columns marked “#ŷ > 7”
list the number of binding scores found “highly
active” for the corresponding receptor.

| | Swiss-Prot | #ŷ > 7 | | |
|---|---|---|---|---|
| No. | accession | (%) | CAS No. | ŷ |
| 1 | P04201 | 712 | 24116-23-2 | 7.68 |
| | | (2.05) | 81382-09-4 | 7.63 |
| | | | 727-81-1 | 7.58 |
| 2 | P35410 | 2476 | 24116-23-2 | 8.26 |
| | | (7.12) | 81382-09-4 | 8.12 |
| | | | 35956-47-9 | 8.07 |
| 3 | Q99788 | 494 | 24116-23-2 | 7.71 |
| | | (1.42) | 81382-09-4 | 7.52 |
| | | | 57718-77-1 | 7.47 |
| 4 | Q99527 | 121 | 24116-23-2 | 7.52 |
| | | (0.35) | 63362-26-5 | 7.30 |
| | | | 24996-74-5 | 7.29 |
| 5 | P32249 | 0 | | |
| 6 | O60883 | 1240 | 81382-09-4 | 7.65 |
| | | (3.57) | 24116-23-2 | 7.63 |
| | | | 6630-44-0 | 7.59 |
| 7 | O14626 | 17 | 15093-31-9 | 7.13 |
| | | (0.05) | 81382-09-4 | 7.12 |
| | | | 35956-47-9 | 7.12 |
| 8 | Q9BXB1 | 169 | 24116-23-2 | 7.32 |
| | | (0.49) | 40323-42-0 | 7.27 |
| | | | 81382-09-4 | 7.26 |
| 9 | P25106 | 420 | 24116-23-2 | 7.53 |
| | | (1.21) | 81382-09-4 | 7.49 |
| | | | 79005-55-3 | 7.45 |
| 10 | Q9BZJ8 | 365 | 81382-09-4 | 7.37 |
| | | (1.05) | 40323-42-0 | 7.32 |
| | | | 70492-71-6 | 7.31 |
| 11 | P46091 | 1795 | 24116-23-2 | 7.80 |
| | | (5.16) | 17304-96-0 | 7.72 |
| | | | 17304-95-9 | 7.72 |
| 12 | P46089 | 3675 | 24116-23-2 | 8.38 |
| | | (10.57) | 81382-09-4 | 8.28 |
| | | | 40323-42-0 | 8.20 |
| 13 | P46093 | 285 | 24116-23-2 | 7.47 |
| | | (0.82) | 6630-44-0 | 7.35 |
| | | | 6630-45-1 | 7.35 |
| 14 | P49683 | 58 | 24116-23-2 | 7.46 |
| | | (0.17) | 81382-09-4 | 7.31 |
| | | | 35956-47-9 | 7.25 |
| 15 | P49685 | 0 | | |
| 16 | Q14330 | 3958 | 81382-09-4 | 8.15 |
| | | (11.39) | 24116-23-2 | 8.11 |
| | | | 35956-47-9 | 8.08 |
| 17 | Q15760 | 138 | 81382-09-4 | 7.29 |
| | | (0.40) | 40323-42-0 | 7.28 |
| | | | 24116-23-2 | 7.26 |
| 18 | Q99678 | 2 | 24116-23-2 | 7.14 |
| | | (0.01) | 81382-09-4 | 7.03 |
| 19 | Q99679 | 1270 | 24116-23-2 | 7.86 |
| | | (3.65) | 81382-09-4 | 7.78 |
| | | | 15093-31-9 | 7.75 |
| 20 | Q99680 | 88 | 24116-23-2 | 7.38 |
| | | (0.25) | 81382-09-4 | 7.30 |
| | | | 17304-95-9 | 7.26 |
| 21 | Q8NDV2 | 0 | | |
| 22 | Q9NS67 | 7 | 24116-23-2 | 7.11 |
| | | (0.02) | 81382-09-4 | 7.11 |
| | | | 40323-42-0 | 7.05 |
| 23 | O00270 | 0 | | |
| 24 | O75388 | 0 | | |
| 25 | O88416 | 7 | 81382-09-4 | 7.13 |
| | | (0.02) | 24116-23-2 | 7.11 |
| | | | 40323-42-0 | 7.07 |
| 26 | Q9UPC5 | 2265 | 24116-23-2 | 8.22 |
| | | (6.52) | 81382-09-4 | 8.17 |
| | | | 5408-02-6 | 8.04 |
| 27 | Q9HC97 | 1060 | 24116-23-2 | 7.70 |
| | | (3.05) | 81382-09-4 | 7.60 |
| | | | 40323-42-0 | 7.57 |
| 28 | O43194 | 214 | 24116-23-2 | 7.47 |
| | | (0.61) | 81382-09-4 | 7.33 |
| | | | 24996-74-5 | 7.31 |
| 29 | O14842 | 850 | 24116-23-2 | 7.73 |
| | | (2.44) | 81382-09-4 | 7.66 |
| | | | 40323-42-0 | 7.58 |
| 30 | O14843 | 0 | | |

TABLE 7
oGPCRs and Predicted High-Affinity Ligands. Targets are
identified by number and Swiss-Prot accession, providing
cross-reference to Table 4 Columns marked “#ŷ > 7”
list the number of binding scores found “highly active”
for the corresponding receptor.

| | Swiss-Prot | #ŷ > 7 | | |
|---|---|---|---|---|
| No. | accession | (%) | CAS No. | ŷ |
| 31 | Q9Y5Y3 | 3011 | 727-81-1 | 7.94 |
| | | (8.66) | 81382-09-4 | 7.91 |
| | | | 6630-45-1 | 7.91 |
| 32 | Q9Y2T5 | 716 | 24116-23-2 | 7.65 |
| | | (2.06) | 81382-09-4 | 7.64 |
| | | | 24996-74-5 | 7.55 |
| 33 | Q9P1P4 | 1325 | 24116-23-2 | 7.76 |
| | | (3.81) | 81382-09-4 | 7.71 |
| | | | 40323-42-0 | 7.69 |
| 34 | Q9BZJ7 | 282 | 81382-09-4 | 7.43 |
| | | (0.81) | 35956-47-9 | 7.42 |
| | | | 24116-23-2 | 7.41 |
| 35 | Q96P68 | 817 | 24116-23-2 | 7.72 |
| | | (2.35) | 81382-09-4 | 7.57 |
| | | | 17304-96-0 | 7.53 |
| 36 | Q96P67 | 22 | 24116-23-2 | 7.15 |
| | | (0.06) | 6630-44-0 | 7.15 |
| | | | 6630-45-1 | 7.15 |
| 37 | Q9H1C0 | 15 | 81382-09-4 | 7.15 |
| | | (0.04) | 24116-23-2 | 7.11 |
| | | | 35956-47-9 | 7.07 |
| 38 | Q96P66 | 437 | 24116-23-2 | 7.44 |
| | | (1.26) | 81382-09-4 | 7.41 |
| | | | 6630-45-1 | 7.36 |
| 39 | Q8TDV0 | 0 | | |
| 40 | Q8TDT2 | 1205 | 24116-23-2 | 7.87 |
| | | (3.47) | 63362-26-5 | 7.69 |
| | | | 81382-09-4 | 7.60 |
| 41 | Q9UJ42 | 872 | 81382-09-4 | 7.63 |
| | | (2.51) | 24116-23-2 | 7.61 |
| | | | 35956-47-9 | 7.56 |
| 42 | Q8N6U8 | 0 | | |
| 43 | Q93126 | 1415 | 24116-23-2 | 7.83 |
| | | (4.07) | 81382-09-4 | 7.75 |
| | | | 40323-42-0 | 7.69 |
| 44 | Q10904 | 11 | 24116-23-2 | 7.22 |
| | | (0.03) | 5408-02-6 | 7.17 |
| | | | 24996-74-5 | 7.14 |
| 45 | Q10907 | 0 | | |
| 46 | Q09965 | 38 | 727-81-1 | 7.13 |
| | | (0.11) | 24116-23-2 | 7.11 |
| | | | 17304-96-0 | 7.09 |
| 47 | Q09966 | 117 | 57718-77-1 | 7.20 |
| | | (0.34) | 24116-23-2 | 7.19 |
| | | | 35878-52-5 | 7.19 |
| 48 | Q11082 | 0 | | |
| 49 | P34311 | 53 | 24116-23-2 | 7.36 |
| | | (0.15) | 81382-09-4 | 7.24 |
| | | | 35878-52-5 | 7.16 |
| 50 | Q03566 | 0 | | |
| 51 | Q18775 | 635 | 40323-42-0 | 7.54 |
| | | (1.83) | 81382-09-4 | 7.52 |
| | | | 24116-23-2 | 7.48 |
| 52 | Q18904 | 0 | | |
| 53 | P34488 | 2217 | 24116-23-2 | 8.05 |
| | | (6.38) | 81382-09-4 | 8.01 |
| | | | 40323-42-0 | 7.91 |
| 54 | Q09638 | 2546 | 24116-23-2 | 8.16 |
| | | (7.33) | 81382-09-4 | 8.09 |
| | | | 40323-42-0 | 8.08 |
| 55 | Q03613 | 0 | | |

1. A method of using a trainable system to predict biomolecular interactions comprising the steps of:
inputting the primary structure of a first set of biomolecules and the structure of ligands having known interactions as a training set into the trainable system,
creating a statistical decision function which recognizes the biomolecular interactions in the training set,
inputting the primary structure of a second set of biomolecules and the structure of ligands of unknown interactions into the statistical decision function, and
outputting predictions from the statistical decision function which predicts interactions between members of the set of unknown interactions.

2. The method of claim 1, wherein the biomolecular interactions are pairwise.

3. The method of claim 2 wherein the pairwise biomolecular interaction comprises specific binding propensities between GPCR and the ligands.

4. The method of claim 1 wherein the ligands include peptides.

5. The method of claim 1, wherein the trainable system is a support vector machine.

6. The method of claim 1, wherein the trainable system is a classification and regression analysis.

7. The method of claim 1, wherein the ligands of the first set of biomolecules and the ligands of the second set of biomolecules are different.

8. The method of claim 1, wherein the ligands of the first set of biomolecules and the ligands of the second set of biomolecules are the same.

9. The method of claim 1 further comprising a step of validating the outputted predictions.

10. The method of claim 9 wherein the validating comprises the steps of:
a. assaying biomolecular interactions between one or more members of the second set of biomolecules and a set of ligands, and
b. comparing the interactions measured in step (a) with the predicted interactions.

11. The method of claim 10 wherein said assaying comprises high throughput screening.

12. The method of claim 10 wherein high-binding ligands are identified.

13. The method of claim 10 wherein cognate ligands are identified.

14. The method of claim 10 wherein said set of ligands comprises a library.

15. The method of claim 10 wherein a library of ligands is identified.

16. The method of claim 1 further comprising the step of designing a ligand from the outputted predictions.

17. The method of claim 16 further comprising the step of validating the design.

18. The method of claim 10 further comprising the step of measuring the bioactivity of ligands.

19. The method of claim 1 wherein said outputted predictions comprise a screen of a set of ligands for biological receptor activity.

20. The method of claim 19 further comprising the step of validating ligands for biological receptor activity.

21. The method of claim 1 further comprising the step of mapping ligands to primary sequence domains of said biomolecules.

22. The method of claim 21 further comprising the step of validating said mapping.
